(19) United States
(12) Patent Application Publication
(10) Pub. No.: US 2026/0142029 A1
(43) Pub. Date: May 21, 2026

(54) METHOD AND APPARATUS FOR TRAINING AN ARTIFICIAL INTELLIGENCE ENGINE TO PROVIDE FIRST AND SECOND MEDICAL OPINIONS

(71) Applicant: SecondOpinionExpert, Inc., Westlake Village, CA (US)

(72) Inventors: Mohan Ananda, Westlake Village, CA (US); Ajay Ananda, Westlake Village, CA (US)

(21) Appl. No.: 18/952,895

(22) Filed: Nov. 19, 2024

(51) Int. Cl. G16H 50/20; G16H 10/60; G16H 15/00

(52) U.S. Cl. CPC G16H 50/20; G16H 10/60; G16H 15/00

(57) ABSTRACT

Method, system, apparatus, and computer-readable media for reporting an automated medical opinion, including receiving, via a user interface, patient medical history, diagnostic tests, and current symptoms for a user; identifying, as automated output from a trained model, a diagnostic assessment including one or more of a medical diagnosis, a recommended medical treatment, or an recommended diagnostic medical test based on the patient medical history, diagnostic tests, and current symptoms received via the user interface; sending a report to the user based on the diagnostic assessment; receiving, via a feedback loop, feedback for the diagnostic assessment reported to the user; and updating the trained model based on the feedback.

### INTRODUCTION

The present disclosure relates generally to the field of a system that communicates a medical opinion to a user based a trained model that uses artificial intelligence (AI) or machine learning (ML).

Medical second opinions have traditionally been obtained by seeking out another healthcare professional who reviews the patient's medical records, diagnoses, and tests. This often involves finding the other healthcare professional, setting up a consultation appointment, and waiting for the appointment to meet with the healthcare professional. The ability to obtain the second opinion may be affected by the availability of the healthcare professional. The timing to obtain the second opinion may delay the selection of a treatment option.

### SUMMARY

The following presents a simplified summary of one or more aspects in order to provide a basic understanding of such aspects. The summary is not an extensive overview of all contemplated aspects and neither identifies key or critical elements of all aspects nor delineates the scope of any or all aspects. Its sole purpose is to present some concepts of one or more aspects in a simplified form as an introduction to the more detailed description that is presented later.

Aspects presented here address the rapidly increasing complexity of medical information, the demand for faster turnaround times, and the limitations of human expert availability have created a need for automated systems that utilize an artificial intelligence engine to generate first and/or second medical opinions.

Artificial intelligence can be used in medical diagnostics, including image recognition, predictive modeling, and natural language processing (NLP). Aspects presented herein provide a medical opinion system that augments traditional second opinion systems by automatically processing patient medical records, diagnostic tests, and clinical data to generate high-quality first and second medical opinions.

Aspects presented herein introduce an AI-based system that can automatically generate a medical opinion, either first or second, using patient history, previous medical tests, and the patient's current condition. The system leverages machine learning models, trained on vast datasets of medical records and outcomes, to perform advanced diagnostics and treatment planning autonomously.

Aspects described herein may include a method for generating an automated medical opinion using artificial intelligence, including any of: collecting patient medical history, diagnostic tests, and current symptoms, pre-processing the collected data to standardize and anonymize the data, using a trained AI engine to analyze the data and generate a diagnostic assessment, providing a treatment plan based on evidence-based clinical guidelines, and/or recommending additional tests, where necessary, to refine the diagnosis and treatment plan.

Aspects described herein include an apparatus for generating an automated medical opinion, including a data input module for collecting patient medical data, a pre-processing module for structuring and anonymizing the data, an AI engine for analyzing the data, generating a diagnosis, suggesting a treatment plan, and recommending additional tests, a report generation module to compile and present the findings, and/or a feedback loop that updates the AI models based on new data and clinician feedback.

Aspects include a system for continuous learning and improvement of automated medical opinions, including a feedback mechanism to allow medical professionals to adjust AI-generated reports and submit feedback, and a machine learning model that incorporates new clinical outcomes, research, and patient data to refine future diagnostic and treatment recommendations.

Additional advantages and novel features of aspects of the present invention will be set forth in part in the description that follows, and in part will become more apparent to those skilled in the art upon examination of the following or upon learning by practice thereof.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 is a diagram illustrating example aspects of a medical opinion system, in accordance with various aspects of the present disclosure.

FIG. 2 illustrates an example exchange of data and communication between a user, a medical opinion system, and sources of medical data, in accordance with various aspects of the present disclosure.

FIG. 3 illustrates an example exchange of data and communication between a user, a medical opinion system, and sources of medical data, in accordance with various aspects of the present disclosure.

FIG. 4A is a flowchart illustrating a method for automatically generating and reporting a diagnostic assessment using a trained model, in accordance with various aspects of the present disclosure.

FIG. 4B is a flowchart illustrating a method for automatically generating and reporting a diagnostic assessment using a trained model, in accordance with various aspects of the present disclosure.

FIG. 5 is a flowchart illustrating a method for automatically generating and reporting a diagnostic assessment using a trained model, in accordance with various aspects of the present disclosure.

FIG. 6 is a block diagram of a computer system on which the disclosed system and method can be implemented, in accordance with various aspects of the present disclosure.

FIG. 7 illustrates an example network environment in which the medical opinion system may be implemented, in accordance with various aspects of the present disclosure.

### DETAILED DESCRIPTION

A patient having a medical issue may visit a physician to assess and evaluate their medical issue. The physician may inform the patient of their opinion as to the cause of the medical issue and/or a proposed treatment plan to address the medical issue. A patient may seek a second opinion from an additional physician. A second opinion may be sought as confirmation of the diagnosis, to see is less invasive and/or alternative options exist for treatment. Medical second opinions have traditionally been obtained by seeking out another healthcare professional who reviews the patient's medical records, diagnoses, and tests. It can be challenging to seek timely, expert medical advice for consultations. For example, the patient may first attempt to identify a healthcare professional with the expertise to provide the second opinion, and then obtaining the second opinion may be delayed based on the healthcare professional's availability and schedule.

U.S. Pat. No. 10,403,395, the entire contents of which are incorporated by reference herein, describes a system that generates a second medical opinion. Aspects presented herein provide improvements that address the rapidly increasing complexity of medical information, the demand for faster turnaround times, and/or the limitations of human expert availability by providing for automated systems that utilize artificial intelligence (AI) and/or machine learning (ML) to generate and report medical opinions.

The present disclosure relates to systems and methods for providing medical opinions in a more comprehensive and efficient manner. Aspects presented herein provide for an apparatus, system, method, and computer-readable medium storing instructions for reporting to users an objective report that includes a medical first and/or second opinion that is obtained, at least in part, using trained artificial intelligence (AI) algorithms. The aspects presented herein leverage patient medical history, diagnostic tests, and/or current medical issues to generate the report that includes diagnostic assessment(s), suggested treatment plan(s), and/or recommendation(s) for additional tests. Some aspects may include automatically generating the medical first and/or second opinion by employing the AI algorithms.

Artificial intelligence including image recognition, predictive modeling, and/or natural language processing (NLP) may be used in connection with medical diagnostics. Such advancements present an opportunity to augment traditional second opinion systems by automatically processing patient medical records, diagnostic tests, and/or clinical data to generate high-quality first and/or second medical opinions.

The present disclosure introduces an AI-based system that can automatically generate a medical opinion, either first or second medical opinions, using patient history, previous medical tests, and/or the patient's current condition. The system leverages machine learning models, which are trained on vast datasets of medical records and outcomes, to perform advanced diagnostics and treatment planning in an autonomous manner that maintains patient privacy.

The system presented herein can provide any combination of an automated diagnosis, a suggested treatment plan, and/or recommendations for additional tests. The system may apply continuous learning to improve the system and keep the analysis current.

For example, an automatic diagnosis may be output based on input of a patient's medical history, symptoms, and/or diagnostic tests. The AI engine (e.g., AI model) may analyze the input data to generate, e.g., output, a diagnosis. If the output of the diagnosis is used as a second opinion, the output may enable a patient to assess a first opinion diagnosis that they may have received from a physician. If the diagnosis is used as a first opinion, the output may guide the patient in seeking treatment with a physician or may guide the physician in selecting additional tests for the patient,

As an example for the suggested treatment plan, the AI engine may provide evidence based treatment recommendations. As the system receives training information, and/or feedback, in an ongoing or continuous manner, the system can continuously evaluate the outcome of various treatment options in order to report a suggestion of a most effective treatment plan based on current evidence.

As an example of recommendations for additional tests, the system may identify one or more additional tests that may be helpful in refining a diagnosis and/or a treatment plan.

The system integrates new medical knowledge, such as the latest research and/or clinical guidelines, in order to keep the training of the AI model up to date. The continuous learning enables the diagnostic assessments of the system to improve over time in an ongoing manner and to keep pace with current medical updates.

The invention also addresses privacy concerns, as all patient data is anonymized and processed in a secure environment, complying with regulations such as Health Insurance Portability and Accountability Act (HIPAA).

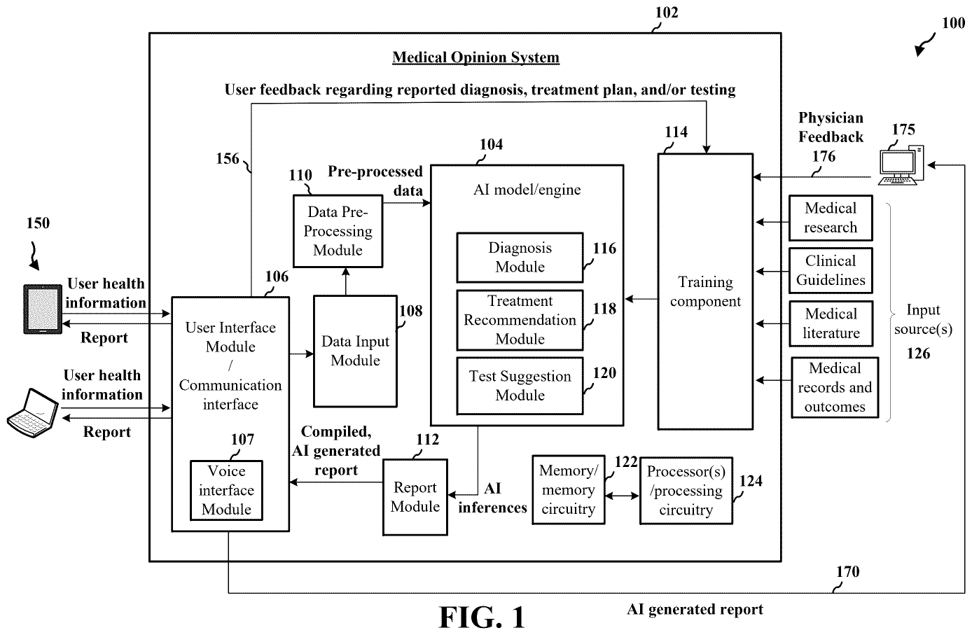

FIG. 1 illustrates an example medical opinion system 102 in accordance with the aspects presented herein. As illustrated, the medical opinion system 102 may include memory 122 (or memory circuitry) and one or more processors 124 (or processor circuitry) configured to cause a computer system to perform the aspects described in connection with the use of the medical opinion system, as described herein.

The medical opinion system 102 includes a data input module 108 that is configured to receive, or collect, all relevant patient information in order to report a medical opinion or suggestions to the user. For example, the data input module 108 receives, or collects, a user's medical history, diagnostic test results (such as blood tests, urine tests, imaging studies (e.g., X-ray information, ultrasound information, CT-scans information, MRI information), and/or biopsy information, among other examples of diagnostic test results), and/or information listing current symptoms. The user data may be received from individual users via a user device 150.

The medical opinion system 102 may include a user interface module 106 that provides a user interface having one or more entry components for a user to enter their health information. For example, the communication interface may provide for communication between the user device 150 and system 102 over a network. For example, the user interface may include one or more entry components that allow a user to type or enter text about symptoms or to select symptoms or areas of concern via a drop down box. In some aspects, the medical opinion system 102 may include a voice module 107 or voice interface (e.g., as part of a user interface or communication interface) that is configured to receive voice entry of data. For example, a user may vocally describe their symptoms, their family history, and/or a diagnosis, among other examples of user information. The medical opinion system may receive the spoken description and convert the description to text and/or process the vocal description for entry to the AI engine 104. A user may enter additional health information, health history information, family history, and/or test result information via an entry component that may allow the user to enter text, select from a set of options, vocally describe information, and/or upload or link files. In some aspects, the user interface may include an entry component that enables a user to upload files or to link files/records for the user.

To assist the user in entering symptom information as part of the input data, the user interface may present the user with a set of categories. The categories help the system narrow the user's medical concern. Once the user has selected a category, the system may start generating questions to analyze the user's medical concern. The system continues to generate questions until the system has exhausted all of its analysis. As the patient answers questions from the system, the system generates questions based on the patient's responses and based on similar data stored in the system regarding similar medical conditions of other patients. The user interface may prompt the user to upload medical documents that he has received from his physician. For example, the user may have digital or paper documents relating to the user's physician's medical evaluation report. In addition, the user may have diagnostic labs, imaging tests, or other tests that may have been performed by his previous physician. If the user has digital copies of these documents, the user can upload the documents to the system. If the user does not have a digital copy of the document, the user can scan or fax the document electronically and the system will store the documents in the user's account.

Any document that is uploaded, or transmitted electronically to the system, will remain confidential and the system will not reveal it to anyone other than the patient and the doctors necessary for the second opinion. The documents may be encrypted or may be automatically deleted after the second opinion process is complete. The only individual that may have access to the user's medical data is the physician reviewing the second opinion report generated by the system.

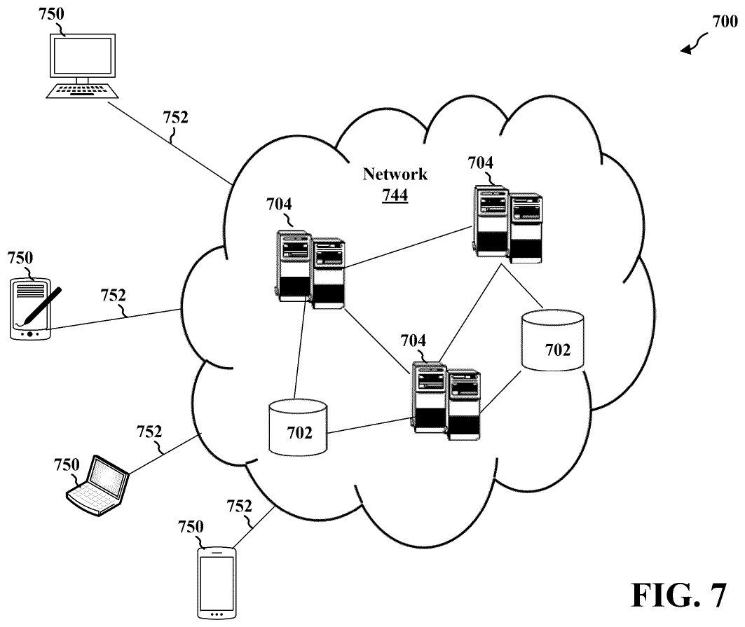

The user interface may also be designed to present AI generated reports, as shown at 170, for display to physicians and/or medical professionals (e.g., at remote device 175, for the physicians/medical professionals to review the AI-generated reports and provide feedback or adjustments, as shown at 176. The user interface with the medical professionals is designed to allow clinicians to interact with the AI suggestions, accepting or modifying the diagnosis or treatment plans as needed. The medical professional feedback, at 176, can be used to refine the AI/ML model or algorithms of the AI engine 104. The user interface module 106 may support multiple input formats such as Electronic Health Records (EHR), PDFs, and/or direct inputs from medical devices. The user interface module 106 may comprise, or be comprised in, a communication interface that is configured to enable the exchange of communication via a communication path (e.g., whether a wired path, a cable path, a fiber optic path, a wireless link, and/or other communication channel between computer systems) between a user device 150 and the medical opinion system. In some aspects, the medical opinion system may be provided at a remote server that communicates with the user devices via the internet, e.g., such as described in connection with FIG. 7.

The medical opinion system 102 may include a pre-processing module 110 that processes raw input data from data input module to prepare the data to be input to the AI engine 104. For example, the pre-processing module 110 may process the raw input data using NLP techniques and/or data normalization to prepare the data to be used as input to the AI engine 104. The patient records, e.g., information, may be processed to translate the records into a structured format that is compatible with the AI engine 104. In some aspects, the pre-processing module 110 may be configured to process vocal description spoken by the user to obtain the relevant description for assessment by the AI engine 104. The pre-processing module 110 may then structure the information from the relevant description into a format that is compatible with the AI engine 104. The pre-processing module 110 may remove or filter irrelevant data before inputting the processed data to the AI engine 104. In some aspects, the pre-processing module 110 may anonymize patient identifiers to ensure patient privacy as the patient information is input to the AI engine 104. The system is a web-based HIPAA compliant and secure system that allows patients a convenient way to register and upload their medical information, test results and diagnostic images. The system is designed to ensure compliance with the HIPAA encryption standards to ensure the security and privacy of an individual's Protected Health Information (PHI). The system will be updated to support new standards issued by the National Institute of Standards and Technology (NIST) regarding the HIPAA encryption standards. Aspects may include establishing a secure connection and/or performing a HIPAA security check. The system is a web-based HIPAA compliant and secure system that allows patients a convenient way to register and upload their medical information, test results and diagnostic images. The system is designed to ensure compliance with the HIPAA encryption standards to ensure the security and privacy of an individual's Protected Health Information (PHI). The system will be updated to support new standards issued by the National Institute of Standards and Technology (NIST) regarding the HIPAA encryption standards.

The AI engine 104 may be built on machine learning algorithms, including deep learning and/or natural language processing. For example, the AI engine 104 may use an AI program that includes a machine learning model or an artificial neural network (ANN) model. The AI engine 104 may include a trained AI model that is trained on a vast dataset of medical cases, outcomes, and/or clinical guidelines to make inferences based on the patient health information that is input by the pre-processing module 110 according to patterns or relationships determined in the data input to the AI engine 104. An inference can refer to an output of the AI engine corresponding to a determination, a prediction, an identification, a decision, a recommendation, or a value. For example, based on pattern(s) or relationship(s) identified in the patient health information that is input to the AI engine, the AI engine 104 outputs an inference for one of more of a potential medical diagnosis to consider, a potential treatment recommendation to consider, and/or additional tests to consider to refine a diagnosis. An ML model may include unsupervised learning, supervised learning, and/or semi-supervised learning. Terms such “AI engine,” “ML engine,” AI/ML engine,” “AI/ML model,” “ML model,” “trained ML model,” “ANN,” “model,” “algorithm,” etc. may be used interchangeably.

In some aspects, the AI engine 104 may use any of machine-learning algorithms, deep-learning algorithms, neural networks, reinforcement learning, regression, boosting, and/or advanced signal processing methods for generating first or second medical opinions.

Reinforcement learning is a type of machine learning that involves the concept of taking actions in an environment in order to maximize a reward. Reinforcement learning is a machine learning paradigm. Other paradigms include supervised learning and unsupervised learning. Basic reinforcement may be modeled as a Markov decision process (MDP) with a set of environment states and agent states, as well as a set of actions of the agent. A determination may be made about a likelihood of a state transition based on an action and a reward after the transition. The action selection by an agent may be modeled as a policy. The reinforcement learning may enable the agent to learn an optimal, or nearly-optimal, policy that maximizes a reward. Supervised learning may include learning a function that maps an input to an output based on example input-output pairs, which may be inferred from a set of training data, which may be referred to as training examples. The supervised learning algorithm analyzes the training data and provides an algorithm to map to new examples.

Regression analysis may include statistical analysis to estimate the relationships between a dependent variable (e.g., an outcome variable) and one or more independent variables. Linear regression is an example of a regression analysis. Non-linear regression models may also be used. Regression analysis may include estimating, or determining, relationships of cause between variables in a dataset.

Boosting includes one or more algorithms for reducing variance or bias in supervised learning. Boosting may include iterative learning based on weak classifiers (e.g., that are somewhat correlated with a true classification) with respect to a distribution that is added to a strong classifier (e.g., that is more closely correlated with the true classification) in order to convert weak classifiers to stronger classifiers. The data weights may be readjusted through the process, e.g., related to accuracy.

Among others, examples of machine learning models or neural networks that may be included in the AI engine 104 include, for example, artificial neural networks (ANN); decision tree learning; convolutional neural networks (CNNs); recurrent neural networks (RNNs), deep learning architectures in which an output of a first layer of neurons becomes an input to a second layer of neurons; support vector machines (SVM), e.g., including a separating hyperplane (e.g., decision boundary) that categorizes data; regression analysis; diffusion models; large language models (LLMs); Bayesian networks; genetic algorithms; deep convolutional networks (DCNs) configured with additional pooling and normalization layers; and/or deep belief networks (DBNs).

In some aspects, the AI engine 104 may include an ANN with interconnected group of artificial neurons (e.g., neuron models) as nodes. Neuron model connections may be modeled as weights, in some aspects. The AI engine 104 may provide predictive modeling, adaptive control, and other applications through training via a dataset relating to medical research, clinical guidelines, medical literature, medical records and corresponding outcomes. In some aspects, the AI engine 104 may include a non-linear statistical data model and/or a decision making model. Machine learning may model complex relationships between input data and output information.

The AI engine 104 may include multiple layers and/or operations that may be formed by concatenation of one or more of the referenced operations. Examples of operations that may be involved include extraction of various features of data, convolution operations, fully connected operations that may be activated or deactivated, compression, decompression, quantization, flattening, etc. The term layer may indicate an operation on input data. Weights, biases, coefficients, and operations may be adjusted in order to achieve an output closer to the target output. Weights and biases are examples of parameters of a trained machine learning model. Different layers of a machine learning model may be trained separately.

A variety of connectivity patterns, e.g., including any of feed-forward networks, hierarchical layers, recurrent architectures, feedback connections, etc., may be included in a machine learning model. Layer connections may be fully connected or locally connected. For a fully connected network, a first layer neuron may communicate an output to each neuron in a second layer. Each neuron in the second layer may receive input from each neuron in the first layer. For a locally connected network, a first layer neuron may be connected to a subset of neurons in the second layer, rather than to each neuron of the second layer. A convolutional network may be locally connected and may be configured with shared connection strengths associated with the inputs for each neuron in the second layer. In a locally connected layer of a network, each neuron in a layer may have the same, or a similar, connectivity pattern, yet having different connection strengths. Aspects of the AI engine 104 may be implemented in various types of processing circuits along with memory and corresponding instruction or code. For example, the AI engine 104 may be implemented via one or more hardware circuits, such as, such as one or more central processing units (CPUs), and/or one or more graphics processing units (GPUs). The AI engine and/or various modules of the AI engine 104 may be stored in memory (e.g., 122) and may be accessible to one or more processors 124 to cause the processors to perform the aspects described in connection with the medical opinion system 102.

The training of the AI engine 104 may be performed initially, and/or in an ongoing manner, by a training component 114. The AI engine 104 may be trained, e.g., with supervised learning, unsupervised learning, semi-supervised learning, and/or reinforcement learning. The training component 114 may be configured to provide the training or that facilitates the training of the AI engine 104. During training, the AI engine 104 may be presented with an input of patient health information, test results, and/or biopsy information that the AI engine 104 uses to compute to produce an output of an inferred medical diagnosis, potential treatment plan, and/or recommendation for addition tests. The actual output may be compared to a target output that links patient health information with diagnoses, treatment plans, or added diagnostic tests, and the difference may be used to adjust parameters (e.g., weights, biases, coefficients, etc.) of the AI/ML model in order to provide an output closer to the target output. Before training, the output may not be correct or may be less accurate. A difference between the output and the target output, may be used to adjust weights of a machine learning model to align the output is more closely with the target, e.g., more closely with intended medical diagnosis, treatment plans, and/or recommended testing.

A learning algorithm may calculate a gradient vector for adjustment of the weights. The gradient may indicate an amount by which the difference between the output and the target output would increase or decrease if the weight were adjusted. The weights, biases, or coefficients of the model may be adjusted until an achievable error rate stops decreasing or until the error rate has reached a target level.

The training data may be collected, or input, for the training component 114 to use to train the AI engine 104. In some aspects, the training component 114 may gather training data by accessing and analyzing medical research, clinical guidelines, medical literature, and medical records and their corresponding outcomes (e.g., including medical history, diagnosis, treatment plan, and/or outcome). In some aspects, as part of training, the training data may be classified across different medical specialties. For example, the AI engine may be trained to identify diagnosis across multiple different potential diagnoses and for a combination of input rather than a single task such as image recognition or identification of a single disease. In contrast to such specialized image recognition or consideration of a single disease, the AI engine 104 is trained to perform a holistic, comprehensive review of patient health history, family health history, current symptoms, and/or diagnostic test result information to generate a first or second medical opinion across multiple medical domains. The medical opinion system 102 incorporates the power of AI to provide real-time diagnostics and automated treatment recommendations. The medical opinion system is configured to combine various medical data sources (e.g., historical records, tests, and current symptoms) to offer comprehensive assessments based on an inference from a trained AI engine. The medical opinion system provides a report suggesting treatment plans and/or diagnostic tests based on a wide array of medical conditions, and not limited to a specific field of medicine or analysis of a single type of data. The medical opinion system utilizes continuous learning from real-world diagnosis and/or treatment outcomes, user feedback, and/or updated clinical guidelines or research to refine the AI algorithms and improve the AI generated recommendations over time. The AI system is configured to produce user reports with first or second opinions that integrate historical data, current medical conditions, and predictive diagnostic insights across a broad range of medical domains.

In some aspects, the training component 114 may receive, gather, and/or collect training data in an ongoing manner. Therefore, the training of the AI engine 104 may continue to improve and/or remain current according to current research and guidelines. In some aspects, the training component 114 may continuously receive such input data. In some aspects, the training component 114 may periodically receive or gather the input data for training the AI engine 104. The input data for training may be collected online and/or offline. Once the AI engine 104 has been trained, further input data, as well as feedback either from a user (e.g., patient) 150 and/or from a medical professional (e.g., in connection with 175) that reviews the inference output by the AI engine may be used to further train, refine, prune, and/or optimize the algorithms of the AI/engine 104.

The AI engine 104 may include a diagnosis module 116 that uses a trained AI algorithm to identify patterns in the patient's data (as input via the user interface module 106), comparing patient symptoms and test results with similar medical cases in its database to generate a preliminary diagnosis as an output from the AI engine 104. The diagnosis module 116 of the AI engine 104 may consider a full medical history of the patient to account for chronic conditions and/or potential comorbidities, for example. If the requested diagnosis is a first opinion, e.g., prior to the patient receiving an opinion from another medical professional, the diagnosis may be inferred separate from, or without, a medical opinion input by the user. If the requested diagnosis is a second opinion, the user may input a prior diagnosis made by a medical professional. In some aspects, the AI engine may consider the prior diagnosis when inferring the diagnosis. In some aspects, the AI engine may infer a diagnosis separate from the prior medical opinion. In some aspects, the AI engine may compare the separate diagnosis to the prior diagnosis.

The AI engine 104 may include a treatment recommendation module 118 that used the trained AI model to infer a recommended treatment plan according to the diagnosis determined by the diagnosis module 116. For example, after forming a diagnosis, the treatment recommendation module 118 generates a treatment plan using an AI model trained based on the latest clinical guidelines, treatment protocols, and/or historical outcomes of similar medical cases. If the requested treatment options are for a first opinion, e.g., prior to the patient receiving a suggestion of treatment options from another medical professional, the potential treatments may be inferred separate from, or without, treatments input by the user. If the requested treatment options are for a second opinion, the user may input a prior suggestion of treatment options made by a medical professional. In some aspects, the AI engine may consider the prior suggestions when inferring the diagnosis. In some aspects, the AI engine may infer potential treatment options separate from the prior medical opinion. In some aspects, the AI engine may compare the separately obtained treatment options to the prior medical opinion.

The AI engine 104 may include a test suggestion module 120 that is configured to output a suggestion of one or more additional tests to improve the precision of the diagnosis determined by the diagnosis module 116. For example, if the data is insufficient for a confident diagnosis, the AI engine 104 suggests additional tests that could help clarify the diagnosis or improve treatment precision. For example, the diagnosis module 116 may determine, or infer, a confidence rating/level associated with the determined diagnosis. If the confidence rating is below a threshold, the test suggestion module 120 may output one or more additional test suggestions to improve the confidence of the diagnosis. The threshold may be different for different diagnoses, for example.

In some aspects, the AI engine 104 may identify one or more medical professionals having experience or expertise associated with the identified diagnosis, identified treatment(s), and/or recommended additional tests. In some aspects, the AI engine 104 may identify the one or more medical professionals further based on access, such as geographic region of the patient, medical insurance of the patient, and/or whether the medical professional is accepting new patients, among other considerations.

The medical opinion system 102 may include a report generation module 112 that is configured to compile one or more inferences from the AI engine 104 (e.g., including inferred diagnoses, suggested treatments, and/or test recommendations), into a coherent and understandable report to be presented to, e.g., displayed to, the user device 150. In some aspects, the report may be tailored for physicians. For example, the system may assist physicians in making a diagnosis, planning a treatment, and/or identifying additional tests for a particular patient. In some aspects, the report may be tailored for patients. For example, the system may assist a patient in identifying a potential diagnosis and finding a corresponding medical specialist. If the report provides a first opinion, the system may assist a patient in obtaining a timely second opinion after they receive an initial diagnosis and treatment plan from a physician. For example, the report may identify one or more medical professionals having experience or expertise associated with the identified diagnosis, identified treatment(s), and/or recommended additional tests. In some aspects, the medical opinion system 102 may include a communication interface, e.g., such as at 106) that enables the user to directly contact one or more of the identified medical professionals. For example, the communication interface may enable the user, e.g. to request a patient appointment with one of the identified medical professionals, set up a video appointment or chat with a medical professional, initiate a request for insurance authorization for the patient associated with the medical professional, among other examples of linking the patient with a selected medical professional.

In some aspects, the report may relate to wellness and/or health maintenance. In some aspects, the report may include preventative recommendations that may assist with lifestyle improvements to alleviate chronic conditions specific to a particular patient. In some aspects, the report may include recommendations for particular vaccinations as a way to improve the wellness of the patient.

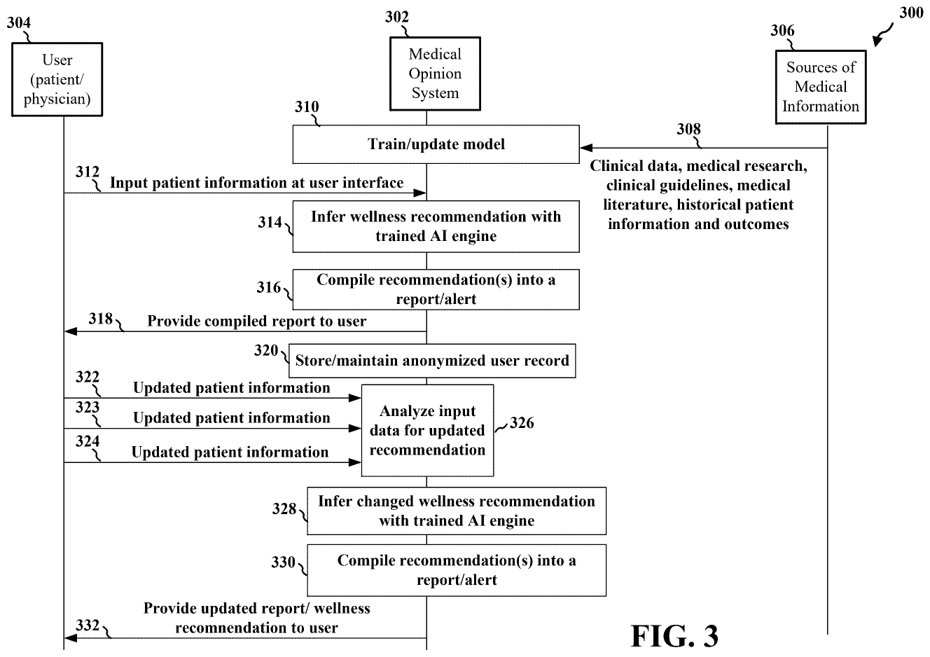

In some aspects, the user may continue to input health information, and the medical opinion system may continue to output personalized medical or lifestyle recommendations that are inferred by the AI engine 104 for improving the patient wellness. As an example, the user, or user device 150, may input ongoing information such as any of heart rate, glucose levels, sleep analysis, weight, blood pressure, and/or symptom levels, among other examples. The AI engine may infer actions or wellness recommendations that would likely improve the health of the patient. For example, the medical opinion system may report a recommendation for the patient to sleep longer, walk more, reduce sugar intake, or make other lifestyle modifications to improve user wellness. FIG. 3 illustrates an example communication flow in which the user may input ongoing updates of patient information and may receive additional reports or recommendations from the medical opinion system.

The medical opinion system 102 incorporates feedback from physicians (e.g., as shown at 176), patients (e.g., as shown at 156), and/or new clinical data (e.g., as shown at 126) into its AI/ML models of the AI engine 104 to allow for continuous learning and continuous refinement of the model or algorithms of the AI engine 104. The continuous learning refers to ongoing reception of updated medical data or feedback, e.g., as such data or feedback becomes available or is received. This continuous learning based on feedback and updated medical information allows the system to evolve, refine, and improve its diagnostic accuracy and treatment recommendations over time.

The medical opinion system 102 may be integrated with various medical insurance carriers such that a patient's bill for services may be sent to the appropriate carrier and/or so that recommended treatments and/or medical professionals may take into consideration the user's medical insurance. In some aspects, the system 102 may also handle disbursements to physicians and patient payment processing options.

The medical opinion system 102 may automatically follow up with users after a period of time following sending the report. The follow up may inquire about the patient, and may seek feedback about the inferences reported to the user. The medical opinion system 102 may use the follow up information from the user to update the user information, or record, in the system and/or to refine the algorithms of the AI engine 104.

Some of the benefits of the medical opinion system include a review and validation of the original diagnoses and opinions and treatment plans, e.g., if the request is for a second opinion, or a diagnosis, treatment plan, or additional test information, that can be provided to the user automatically in an efficient manner. The user does not need to wait to have an appointment with a physician to obtain a first or second opinion and does not need to identify a particular specialist before seeking an first/second opinion. The system 102 is able to analyze general medical information to identify potential concerns for the user and to make an inferred diagnosis, recommend a treatment plan or added tests. The services are available to the user without any need for the patient to travel, for example.

The system 102 may initiate a user registration with the system, e.g., requesting the user to enter personal information, such as his name, birth date and gender. After entering personal information, the user may choose a username and password that is secure and unique to the user. After selecting a username and password, the user may login to access the features of the system 102.

In some aspects, the system 102 may provide a further option for a video, or other, consultation with a physician after the report is provided to the user and may offer options of one or more physicians. If the user selects a consultation with a physician, then the system 102 may provide the user's information to the physician and/or may initiate a videoconference through the system 102. For example, during the video conference, the user may review the report with the physician and ask the physician any questions or concerns he may have regarding the AI generated diagnosis or recommendation in the report that the user received.

In some aspects, the system 102 may coordinate payment between the user and the physician, e.g., and may allow the user to enter medical insurance information into the system so that the system can bill the appropriate insurance on behalf of the patient. In addition, the user may enter a credit card, or bank account information, to keep on file in the event there are expenses that are not covered by the insurance that need to be paid by the user.

If the user selects the option to have a consultation with a physician, the system may present the option for the report and/or medical information to be provided to the selected physician. The system may send all of the information about the user that was obtained from the dialogue between the user and the system to the selected physician. The system may provide secure hyperlinks which link to all of the medical documents that were uploaded or transmitted electronically to the system. The secure hyperlinks allow the physician reviewing the report to conveniently access the user's additional medical documents. A status report may be provided to the consulting physician giving the consulting physician a comprehensive status of the patient's current medical condition that includes information regarding the treatment plan suggested by the system and also includes hyperlinks to all of the medical data that was uploaded to the system. The current status report allows the consulting physician to begin a thorough analysis of the patient's condition, thereby allowing the consulting physician to make a well-informed assessment as to the patient's condition. The system may provide any additional options for consulting with a physician, as described in connection with U.S. Pat. No. 10,403,395, the entire contents of which are incorporated herein by reference.

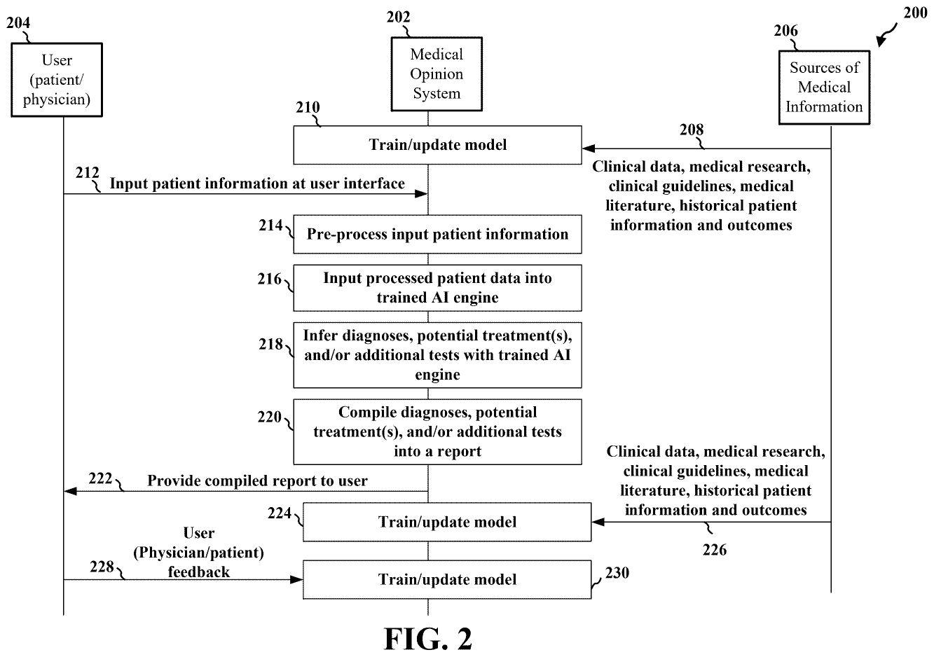

FIG. 2 is a communication flow 200 between a medical opinion system 202 (e.g., which may include one or more aspects of the medical opinion system 102 in FIG. 1) and a user 204 that interacts with the medical opinion system 202 via a user interface, such as described in connection with FIG. 1. In some aspects, the user may be a physician or medical professional entering medical information about a patient. In some aspects, the user may be a person seeking medical opinion information for themselves. The medical opinion system 202 may include a trained AI/ML model, such as described in connection with the AI engine 104. FIG. 2 shows that the medical opinion system 202 may train or update the AI/ML model, or AI/ML algorithms, at 210. The training may be performed based on any combination of clinical data, medical research, clinical guidelines, medical literature, and/or historical patient information and outcomes, among other example of medical information. As shown at 208, the medical opinion system may receive the medical information for training the model from external sources of medical information 206, which may be accessed via remote sources. Once the AI engine is trained, the user 204 inputs patient information at a user interface, at 212, and requests an inferred medical opinion from the medical opinion system 202. The input at 212 may be via the user interface module 106 described in connection with FIG. 1, for example. The entry may be as text, selections of options, upload of files, and/or spoken words. The input medical information may include any combination of patient symptoms, patient medical and family history, diagnostic test results, and/or biopsy information. At 214, the medical opinion system 202 may pre-process the input patient information to structure the received data in a manner to be analyzed by the AI engine. At 216, the pre-processed patient data is input to the AI engine and analyzed based on the trained AI/ML model. At 218, the AI engine infers one or more diagnoses, one or more potential treatments for the identified diagnoses, and/or one or more additional tests that may enable a more accurate diagnosis, such as described in connection with the AI engine 104 in FIG. 1. At 220, the medical opinion system compiles the inferred diagnose(s), potential treatment(s), and/or recommended test(s) into a user report. The report may be compiled differently and or more include different information depending on whether the user that requested the information is a medical professional or the patient themselves. At 222, the complied report is provided to the user. For example, the compiled report may be displayed to the user at the user interface or may be provided in an email, text message, or other manner to the user 204. The AI engine may continue to receive updated training data and feedback to refine the AI/ML model and improve the inferences output by the AI engine. For example, at 224, the medical opinion system may train or update the AI/ML of the AI engine based on additional medical information 226 received from external sources of medical information. For example, this additional medical information may include any combination of updated clinical data, medical research updates, updated clinical guidelines, new medical literature, and/or additional patient information and outcomes, among other example of updated medical information. The medical opinion system 202 may further train or update the AI/ML model at 230 based on feedback 2228 from the user 204. As an example, the feedback from the user 204 may indicate that the inferred diagnosis was correct, that one of the potential treatment plans were followed and was successful in treating the medical condition, and/or that the suggested tests were helpful in confirming the diagnosis. Alternately, the feedback may indicate that the inferred diagnosis was incorrect and may be provided the correct diagnosis as feedback. The feedback may indicate that the inferred treatment plan was not effective and/or that another treatment plan had a better outcome for the medical condition. The feedback may indicate that the recommended tests were not helpful and/or a reason that the tests should not be recommended. The feedback 228 may then be used to update the AI/ML model to further refine the inferences provided by the AI engine of the medical opinion system 202.

FIG. 3 is a communication flow 300 between a medical opinion system 302 (e.g., which may include one or more aspects of the medical opinion system 102 in FIG. 1 and/or the medical opinion system 202 in FIG. 2) and a user 304 that interacts with the medical opinion system 302 via a user interface, such as described in connection with FIG. 1. In some aspects, the user may be a physician or medical professional entering medical information about a patient. In some aspects, the user may be a person seeking medical opinion information for themselves.

The medical opinion system 302 may include a trained AI/ML model, such as described in connection with the AI engine 104 and the medical opinion system 202 in FIG. 2. FIG. 3 shows that the medical opinion system 302 may be trained and updated, at 210 based on ongoing input of clinical data, medical research, clinical guidelines, medical literature, and/or historical patient information and outcomes, among other example of medical information. As shown at 308, the medical opinion system may receive the medical information for training the model from external sources of medical information 306, which may be accessed via remote sources.

The user 304, e.g., user device such as 150, sends patient information to the medical opinion system 302 (e.g., such as input at a user interface). The input medical information may include any combination of patient symptoms, patient medical history, family medical history, diagnostic test results, biopsy information, and/or daily health information (e.g., such as glucose levels, weight, sleep information, heart rate, blood pressure, etc.). The data may be pre-processed and input to an AI engine, such as described at 214 and 216 in FIG. 2. At 314, the AI engine infers one or more diagnoses, one or more potential treatments for the identified diagnoses, and/or one or more additional tests that may enable a more accurate diagnosis, and/or wellness recommendations, such as described in connection with the AI engine 104 in FIG. 1. At 316, the medical opinion system compiles the inferred diagnose(s), potential treatment(s), recommended test(s), and/or wellness recommendations into a user report. At 318, the complied report is sent to, or displayed to the user. For example, the compiled report may be displayed to the user at the user device or may be provided in an email, text message, alert displayed at a website, displayed as part of an application, or in another manner. The AI engine may continue to receive updated training data and feedback to refine the AI/ML model and improve the inferences output by the AI engine. The medical opinion system may receive feedback, e.g., feedback from users and/or medical professionals that may indicate the accuracy of an inferred diagnosis, and/or the effectiveness of the recommended treatment or wellness suggestions. The feedback may be used to refine the AI engine of the medical opinion system 302.

As shown at 322, 323, and 324, the user, or user device, may continue to send updated patient information. For example, the user/user device may send periodic health metric information (e.g., weight, heart rate, glucose levels, blood pressure, etc.). As shown at 320, the medical opinion system may store, and maintain, an anonymized user record that includes the received user health information (e.g., from 312 and later from 322, 323, and 324, as well as any previously reported inferences). The input of information at 312 may include aspects described in connection with 212 in FIG. 2 and/or the entry of information via the user interface module 106 in FIG. 1, for example. The maintained record may be configured to comply with all privacy requirements, including any HIPAA regulations. In some aspects, the record may remove the association of user contact information and/or user identify information from the medical/health information stored in the record. The record may store an record ID as a connectivity point between the user and the user's medical record. As shown at 326, when the medical opinion system 202 receives updated user information, the AI engine may perform an inference to determine if there is an updated wellness recommendation, diagnosis, treatment plan, and/or recommended tests based on the combination of the updated user health information with the stored user health record. If there is no change in the inferences or recommendation(s), the medical opinion system 302 may skip sending a report or message to the user 304, or the medical opinion system 302 may inform the user that there is no change. If there is a new or different inference at the AI engine, as shown at 328, the medical opinion system may compile the new recommendation(s) into a report or alert for the user, and send the wellness recommendation to the user at 332. Although only a single update is shown, at 332, the process may be an ongoing process in which the medical opinion system continues to receive updated health information from the user 304, maintains the user record stored for the user, performs a new analysis via the AI engine, and sends ongoing or updated reports or alerts, e.g., as new inferences are determined by the AI engine.

In some aspects, the user may access the medical opinion system 302 via a website, and the medical opinion system may be provided as a web-based platform. In some aspects, the medical opinion system 302 may provide a user interface to the user as an application at the user's device, such as an application on a smart phone or tablet.

In some aspects, the medical opinion system may provide multiple tiers of service. As an example, a first tier of service may be provided without a subscription and may be freely accessible. The user may enter general information, such as contact information (e.g., name and email address) and demographic information such as age and gender. The medical opinion system may send the user a simplified questionnaire with a subset of medical questions. The medical opinion system may then send a preliminary opinion to the user with any combination of aspects described in connection with FIGS. 1-3. In some aspects, a second tier of service may be based on a purchase, whether one-time or an ongoing subscription to the services of the medical opinion system. The medical opinion system may collect additional medical records for the user, e.g., from the user's physician, and receive additional, more detailed medical and health information about the user. The medical opinion system may then provide a more detailed report for the user. In some aspects, the additional information may include the ongoing medical or health information described in connection with FIG. 3, and the more detailed report may include ongoing wellness recommendations, as described in connection with FIG. 3. In some aspects, the medical opinion system (e.g., 102, 202, and/or 302) may be freely accessibly by physicians and may enable physicians to upload their areas of expertise, their contact information, and/or their schedule information to allow the medical opinion system to identify medical professionals relating to the inferred medical opinion.

The present disclosure introduces a novel AI-based method and apparatus for generating medical opinions, extending the capabilities of medical opinion systems by automating the diagnostic process, offering treatment recommendations, and/or suggesting additional tests using AI/ML models and algorithms. The proposed system improves both the speed and accuracy of first and second medical opinions, while continuously learning from new data and outcomes.

Aspects described herein may include a method for generating an automated medical opinion using artificial intelligence, including any of: collecting patient medical history, diagnostic tests, and current symptoms, pre-processing the collected data to standardize and anonymize the data, using a trained AI engine to analyze the data and generate a diagnostic assessment, providing a treatment plan based on evidence-based clinical guidelines, and/or recommending additional tests, where necessary, to refine the diagnosis and treatment plan.

Aspects described herein include an apparatus for generating an automated medical opinion, including a data input module for collecting patient medical data, a pre-processing module for structuring and anonymizing the data, an AI engine for analyzing the data, generating a diagnosis, suggesting a treatment plan, and recommending additional tests, a report generation module to compile and present the findings, and/or a feedback loop that updates the AI models based on new data and clinician feedback.

Aspects include a system for continuous learning and improvement of automated medical opinions, including a feedback mechanism to allow medical professionals to adjust AI-generated reports and submit feedback, and a machine learning model that incorporates new clinical outcomes, research, and patient data to refine future diagnostic and treatment recommendations.

As one non-limiting example to illustrate the potential benefits of such a medical opinion system, a user may be a parent with a child that wakes up in the middle of the night with moderately intense abdominal pain. The user may have assessed that it is the pain is not bad enough that they need to call an ambulance, but it is concerning enough that the user is considering calling the pediatrician to wake them up for a consultation. The user may be hesitant to inconvenience the doctor unnecessarily. The medical opinion system presented herein enables the user to obtain additional information to make a decision about whether to contact the doctor. For example, the user may use their phone to open an application, answer a few questions to describe their child's current symptoms. The medical opinion may already have the medical history information for the child, and may have access to the child's medical records, in some aspects. The parent may receive an automated potential diagnosis or treatment suggestion that may offer insight on the child's situation with the skill level of a medical specialist. As an example, the application may confirm that the situation is not urgent, but does require further medical attention in the next day or two. The user can forward, via the application, the received report to their child's pediatrician. This way, when the parent contacts the pediatrician's office to make an appointment, they will already have the report for reference. Alternatively, the pediatrician's office may receive the report and contact the user to set up an appointment to follow up on the child's symptoms.

As another example, a patient may have been involved in a car crash and transported to the hospital for emergency care. The patient does not have broken bones, but had an emergency tracheotomy and there are several deep wounds. The surgery goes well, and the patient is in post-op recovery. To avoid unnecessary costs and risks of infection in the hospital, the patient's insurance plan or medical provider may want to move the patient out of the hospital and into a more appropriate post-acute care facility as soon as possible, while keeping in mind the patient's specific needs for tracheotomy and wound care. The medical opinion system may use the patient's personal medical data and insurance plan benefit information as input to the AI engine to identify a facility that meets the care needs and currently has an empty bed. In some aspects, the user may be the patient. In some aspects, the user may be the hospital discharge planner, and the medical opinion system may allow the user to exchange communication with the identified facility in order to facilitate all the necessary paperwork for approvals, as well as transferring the patient's medical records to the facility. This enables the patient to arrive at the new facility with everything in place.

In some aspects, the user may be a medical professional or care provider rather than the patient themselves. The user may use the information provided by the system to improve their medical assessments, treatments, and general medical care. As an example, a particular medical provider or insurance plan may have a history of achieving an overall Medicare Star Rating of 2.5 stars, leading to underperformance during re-enrollment periods, and less-than-desired profitability. By integrating the combination of AI-driven expert opinions to improve diagnosis and treatment planning, medical opinion system may improve medical treatment and overall profitability. The AI-enhanced flow of care with a network of technology enabled care providers may assist the medical provider in adjusting pricing and benefit management based on data analytics on their entire population of customers in order to improve their general rating leading to increased customer enrollment.

As another example, a patient may be retired with a recent knee replacement, Type 2 diabetes, and COPD. A home health agency may be visiting him regularly at home using our computer-based platform that includes or is comprised in the medical opinion system presented herein. The platform may serve as a mobile electronic medical record (EMR), so that the health technicians and therapists have access to the patient's medical record and can update it with each visit. The patient may also be enrolled in, or subscribed to, a program for constant (e.g., 24/7) monitoring relating to his diabetes and COPD. The patient's medical information may be constantly reviewed behind the scenes by the medical opinion system to automatically notify the patient, and/or his physician, of any significant changes in his health status.

As the number of personal health records obtained through expert opinions, medical record flow for patient care, and whole-genome sequencing and interpretation increases, the medical opinion system refines the trained model to improve medical assessments and improve patient outcomes. For example, as the medical opinion system receives millions of personal health records, assesses diagnoses, treatments, and corresponding outcomes, as well as receives feedback about prior diagnoses, testing, and treatment suggestions, the medical opinion system improves the accuracy of the output from the trained model of the AI engine. The medical opinion system can work in connection with a robust network of care providers and payers to conduct big data analytics to improve best practices for health care decision making and delivery of care, as well as to stimulate new health discoveries.

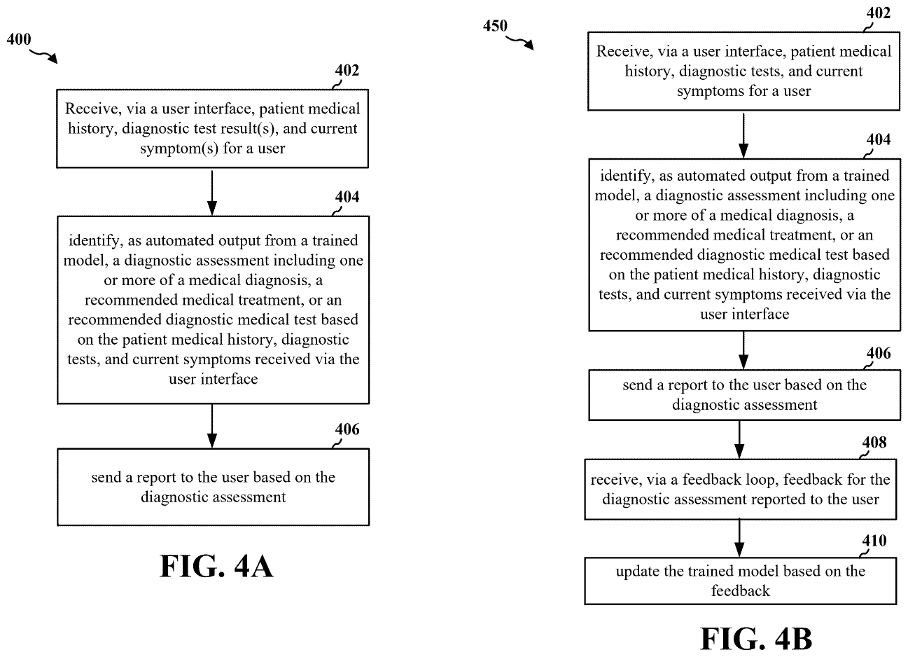

FIG. 4A is a flowchart 400 of a method of automatically generating a medical diagnostic assessment using a trained model. The method may be performed at a medical opinion system (e.g., 102, 202, or 302). Some aspects of the method may be performed by an AI engine 104 or AI engine component 475 of a processing system that may be part of a medical opinion system.

As shown at 402, the medical opinion system receives, via a user interface, patient medical history, diagnostic tests, and/or current symptom information for a user. The user may be a person requesting first or second medical opinion for themselves. In some aspects, the user may be referred to as the patient. The user may be a physician or medical professional that inputs information for a patient. For example, the information may be received from a user device 150 via the user interface module 106 of the medical opinion system 102. FIG. 2 and FIG. 3 illustrate examples of a user sending patient information, at 212 and 312, for example.

In some aspects, the medical opinion system may receive, via the user interface at 402, at least one of the patient medical history or the current symptom information for the user as spoken words via a voice module. In some aspects, the medical opinion system may further identify relevant medical information in the spoken words received via the voice module and structure the relevant medical information from the spoken words in a format for entry as input to the trained model to obtain the diagnostic assessment from the trained model.

As illustrated at 404, the medical opinion system generates (e.g., identifies, infers, determines, or decides), as automated output from a trained model, a diagnostic assessment including one or more of a medical diagnosis, a recommended medical treatment, or an recommended diagnostic medical test based on the patient medical history, diagnostic tests, and current symptoms received via the user interface. For example, one or more medical diagnoses may be inferred by the diagnosis module 116 of the AI engine 104 in FIG. 1. As another example, one or more recommended treatments for the inferred diagnoses may be inferred by the treatment recommendation module 118 of the AI engine 104 in FIG. 1. As another example, one or more recommended diagnostic tests may be inferred by the test suggestion module 120 of the AI engine 104 to refine the diagnosis and/or treatment recommendations. As an example, the trained model may an artificial intelligence model, e.g., such as the AI engine 104 described in connection with FIG. 1. The AI model may be trained for medical diagnostic assessments across multiple medical areas (e.g., various categories, various specialties, various potential medical conditions, various potential treatment options, various types of input and clinical tests) based on a combination of medical cases, medical research, clinical guidelines, and historical patient outcomes. As an example, the model may be trained to consider input of various test results (e.g., blood test results, urine test results, images or analysis from ultrasound, CT-scan, MRI, X-rays, biopsy results, among other examples) in combination with symptom description and medical history to output an inference of one or more diagnoses, treatments, or additional test recommendations.

As illustrated at 406, the medical opinion system may send a report to the user based on the diagnostic assessment (e.g., the report including one or more of the diagnosis, recommended treatment(s), and/or recommended test(s) inferred by the AI engine). The report may be compiled by the report generation module 112 and sent via the user interface module 106, for example. FIGS. 2 and 3 illustrate examples of reports being set to the user, e.g., at 222, 318, and/or 332.

FIG. 4B is a flowchart 450 of a method of automatically generating a medical diagnostic assessment using a trained model. The method may be performed at a medical opinion system (e.g., 102, 202, or 302). Some aspects of the method may be performed by an AI engine 104 or AI engine component 475 of a processing system that may be part of a medical opinion system. The aspects of FIG. 4B that have been described in connection with FIG. 4A are shown with a same reference number.

FIG. 4B illustrates that the medical opinion system may receive, via a feedback loop, feedback for the diagnostic assessment reported to the user. The feedback may be received from the user (e.g., whether a patient or physician). FIG. 1 illustrates an example at 156 showing feedback from a user. In some aspects, the medical opinion system may send the diagnostic assessment to one or more medical professionals, and the feedback may be received from the one or more medical professionals indicating an adjustment to the diagnostic assessment. FIG. 1 illustrates an example showing feedback (e.g., 176) from medical professionals.

At 410, the medical opinion system updates the trained model based on the feedback. For example, FIG. 2 illustrates an example of feedback, at 228, that is used to further train, update, or refine the model or algorithms of the medical opinion system 202 (e.g., of the AI engine 104). For example, refining the model may include adjusting weights or biases of the model to align the output of the model more closely with an intended output based on the feedback.

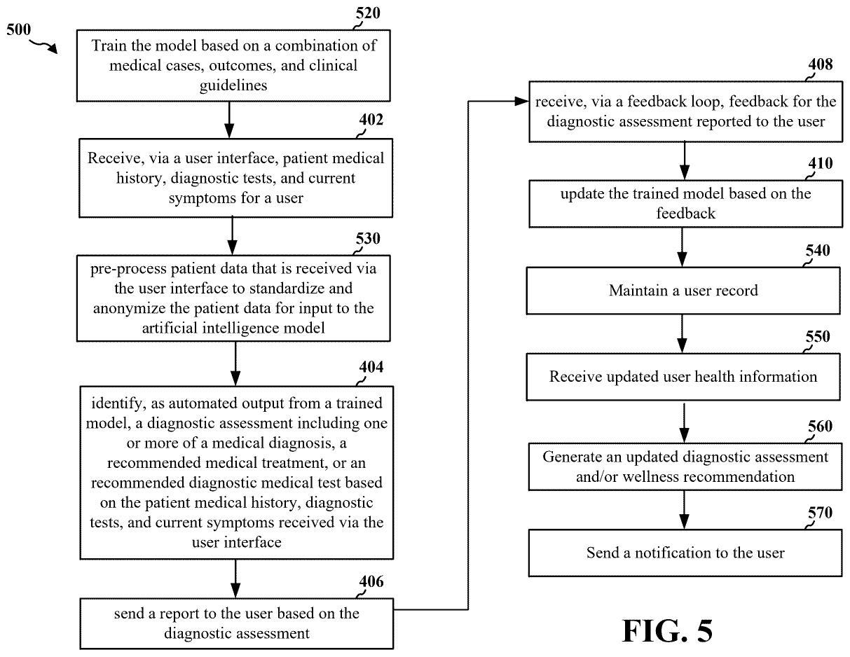

FIG. 5 is a flowchart 500 of a method of automatically generating a medical diagnostic assessment using a trained model. The method may be performed at a medical opinion system (e.g., 102, 202, or 302). Some aspects of the method may be performed by an AI engine 104 or AI engine component 475 of a processing system that may be part of a medical opinion system. The aspects of FIG. 5 that have been described in connection with FIG. 4A and/or FIG. 4B are shown with a same reference number.

As shown at 520, the medical opinion system may train the model based on a combination of medical cases, outcomes, and clinical guidelines before receiving the user information at 402. The AI model may be trained for medical diagnostic assessments across multiple medical areas (e.g., various categories, various specialties, various potential medical conditions, various potential treatment options, various types of input and clinical tests) based on a combination of medical cases, medical research, clinical guidelines, and historical patient outcomes. For example, rather that analyzing a particular type of input (such as an image) for the presence or absence of a single medical condition, the model is trained to provide a comprehensive inference across various potential medical diagnoses and using a combination of different types of input data. For example, the training may be performed by the training component 114 in FIG. 1.

The training may be an ongoing training that is continually (e.g., periodically or as updated training information becomes available) performed. For example, the medical opinion system may receive updated training data that includes one or more of: additional (e.g., new, recent, or updated) medical research, updated clinical guidelines, or additional medical cases and/or patient outcome information. In some aspects, the added training material may be input to the medical opinion system. In some aspects, the medical opinion system may continuously search for updated training material. Then, the medical opinion system may refine the model based on the updated training data. FIG. 2 illustrates examples of the medical opinion system (e.g., the AI engine or model) being updated, trained, or refined based on the additional information received/input at 226

As shown at 530, the medical opinion system may pre-process patient data that is received via the user interface to standardize and anonymize the patient data for input to the artificial intelligence model. For example, the pre-processing may be performed by the module 110 in FIG. 1. In some aspects, the medical opinion system may receive, via the user interface at 402, at least one of the patient medical history or the current symptom information for the user as spoken words via a voice module. In some aspects, the medical opinion system may further identify relevant medical information in the spoken words received via the voice module and structure the relevant medical information from the spoken words in a format for entry as input to the trained model to obtain the diagnostic assessment from the trained model. In some aspects, the analysis and structuring may be performed as part of the pre-processing at 530.

As illustrated at 540, the medical opinion system may maintain a user record with collected information and prior diagnostic assessment. FIG. 3 illustrates an example, at 320, of the store or maintenance of an anonymized user (or patient) record.

At 550, the medical opinion system receives, via the user interface, updated user health information. For example, FIG. 3 illustrates that updated patient information may be sent at 322, 323, 324, etc.

At 560, the medical opinion system generates an updated diagnostic assessment or wellness recommendation for the user from the trained model based on the user record and the updated user health information. FIG. 3 illustrates an example at 326 and/or 328.

At 570, the medical opinion system sends a notification to the user of the updated diagnostic assessment or the wellness recommendation generated by the trained model. The notification may be an updated report, an alert, a text, a message, or another indication of the updated diagnostic assessment. FIG. 3 illustrates an example, at 332, of a wellness recommendation that is sent to the user.

In some aspects, the receipt of the initial report, and/or ongoing notifications or updates or wellness recommendations may be based on a user subscription to the medical opinion system. In such examples, the medical opinion system may receive a user subscription to ongoing diagnostic assessments or wellness recommendations. The subscription may be received prior to providing an initial assessment and/or prior to providing the user with ongoing notifications.

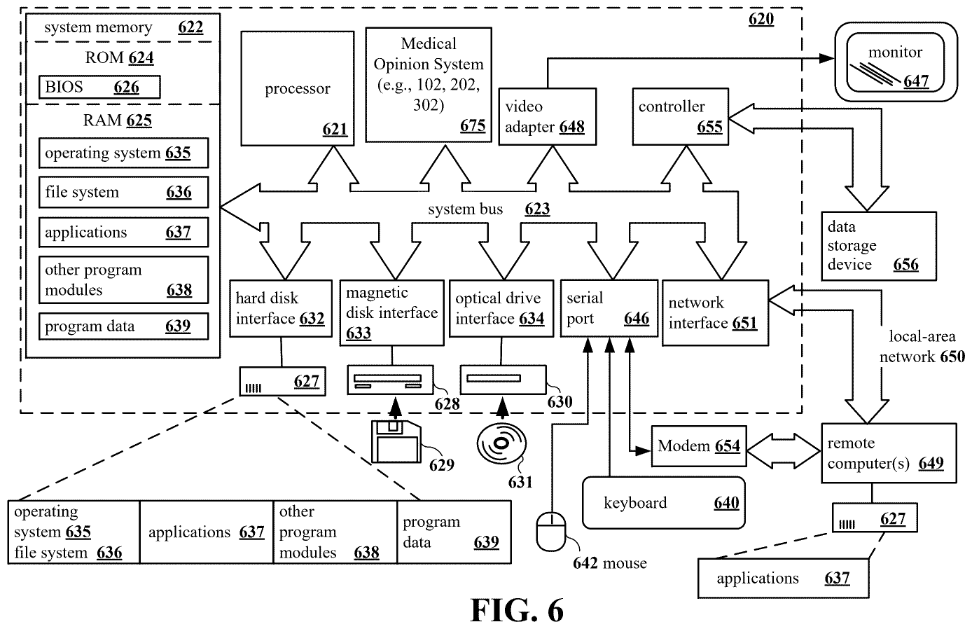

FIG. 6 is a block diagram illustrating a computer system 620 on which aspects of the medical opinion system, e.g., as described in connection with any of FIGS. 1-5 may be implemented in accordance with an example aspect. The computer system 620 can correspond to the physical server(s) on which the medical opinion service (e.g., 102, 202, 302) is executing. In one configuration, computer system 620 may include one or more modules of a medical opinion system 675. The medical opinion system 675 and/or the computer system 620, and in particular, the file system 636 and/or the processor (e.g., 621), may be configured to perform the aspects of the flowcharts in FIGS. 4A, 4B, and/or 5, and or to perform the aspects described in connection with the medical opinion system 102, 202, and/or 302.

As shown, the computer system 620 (which may comprise a personal computer or a server) includes a central processing unit 621, a system memory 622, and a system bus 623 connecting the various system components, including the memory associated with the central processing unit 621. As will be appreciated by those of ordinary skill in the art, the system bus 623 may comprise a bus memory or bus memory controller, a peripheral bus, and a local bus that is able to interact with any other bus architecture. The system memory may include permanent memory (ROM) 624 and random-access memory (RAM) 625. The basic input/output system (BIOS) 626 may store the basic procedures for transfer of information between elements of the computer system 620, such as those at the time of loading the operating system with the use of the ROM 624.

The computer system 620 may also comprise a hard disk 627 for reading and writing data, a magnetic disk drive 628 for reading and writing on removable magnetic disks 629, and an optical drive 630 for reading and writing removable optical disks 631, such as CD-ROM, DVD-ROM and other optical media. The hard disk 627, the magnetic disk drive 628, and the optical drive 630 are connected to the system bus 623 across the hard disk interface 632, the magnetic disk interface 633, and the optical drive interface 634, respectively. The drives and the corresponding computer information media are power-independent modules for storage of computer instructions, data structures, program modules, and other data of the computer system 620.

An example aspect comprises a system that uses a hard disk 627, a removable magnetic disk 629 and a removable optical disk 631 connected to the system bus 623 via the controller 655. Any type of media 656 that is able to store data in a form readable by a computer (solid state drives, flash memory cards, digital disks, random-access memory (RAM) and so on) may also be utilized.

The computer system 620 has a file system 636, in which the operating system 635 may be stored, as well as additional program applications 637, other program modules 638, and program data 639. A user of the computer system 620 may enter commands and information using keyboard 640, mouse 642, or any other input device known to those of ordinary skill in the art, such as, but not limited to, a microphone, controller, scanner, etc. Such input devices typically plug into the computer system 620 through a serial port 646, which in turn is connected to the system bus. Input devices may be also be connected in other ways, such as, without limitation, via a parallel port, a game port, or a universal serial bus (USB). A monitor 647 or other type of display device may also be connected to the system bus 623 across an interface, such as a video adapter 648. In addition to the monitor 647, the computer system may be equipped with other peripheral output devices (not shown), such as loudspeakers, a printer, etc.

Computer system 620 may operate in a network environment, using a network connection to and/or providing a user interface to one or more remote computers 649. The remote computer (or computers) 649 may be local computer workstations or servers comprising most or all of the aforementioned elements in describing the nature of a computer system 620. Other devices may also be present in the computer network, such as, but not limited to, routers, network stations, peer devices or other network nodes. The connections via a network may provide a communication interface that enables the system to receive information from multiple user and to provide medical opinion reports, as described herein, to multiple remote users.

Network connections can form a local-area computer network (LAN) 650 and a wide-area computer network (WAN). Such networks are used in corporate computer networks and internal company networks, and they generally have access to the Internet. In LAN or WAN networks, the computer system 620 is connected to the local-area network 650 across a network adapter or network interface 651. When networks are used, the computer system 620 may employ a modem 654 or other modules that enable communications with a wide-area computer network such as the Internet. The modem 654, which may be an internal or external device, may be connected to the system bus 623 by a serial port 646. The example network connections are non-limiting examples of numerous well-understood ways of establishing a connection by one computer to another using communication modules.

FIG. 7 shows an example communication system 700 usable in connection with aspects described herein for the medical opinion system. The communication system 700 includes one or more accessors (also referred to interchangeably herein as one or more “users,” people, or person) and one or more terminals 750. In one aspect, data for use in accordance aspects presented herein, for example, input and/or accessed by accessors via terminals 750, such as personal computers (PCs), minicomputers, mainframe computers, microcomputers, telephonic devices, or wireless devices, such as personal digital assistants (“PDAs”) or a hand-held wireless devices coupled to one or more servers 704 such as a PC, minicomputer, mainframe computer, microcomputer, or other device having a processor and a repository for data and/or connection to a repository 702 for data, via, for example, a network 744, such as the Internet or an intranet, and couplings 752. The computer system 620 including the medical opinion system 675 may be comprised in the network 744, e.g., including one or more servers 704 or data repositories 702. The data repositories may include storage that provides non-volatile, bulk or long term storage of data or instructions in the medical opinion system. The storage may take the form of a disk, tape, CD, DVD, or other reasonably high capacity addressable or serial storage medium. Multiple storage devices may be provided or available, and some include network storage or cloud-based storage.

The couplings 752 may include, for example, wired, wireless, or fiberoptic links. For example, the couplings may be part of a communications interface with the medical opinion system. Such a communication interface allows software and data to be transferred between the medical opinion system and external devices, e.g., 750. Examples of communications interfaces may include a modem, a network interface (such as an Ethernet card), a communications port, a Personal Computer Memory Card International Association (PCMCIA) slot and card, etc. Software and data transferred via communications interface can be in the form of signals, which may be electronic, electromagnetic, optical or other signals capable of being received by the communications interface. These signals may be provided to communications interface via a communications path (e.g., channel). This path carries signals and may be implemented using wire or cable, fiber optics, a telephone line, a cellular link, a radio frequency (RF) link and/or other communications channels.

In another aspect, the methods and system presented herein may operate in a stand-alone environment, such as on a single terminal.

In various aspects, the systems and methods described herein may be implemented in hardware, software, firmware, or any combination thereof. If implemented in software, the methods may be stored as one or more instructions or code on a non-transitory computer-readable medium. Computer-readable medium includes data storage. By way of example, and not limitation, such computer-readable medium can comprise RAM, ROM, EEPROM, CD-ROM, Flash memory or other types of electric, magnetic, or optical storage medium, or any other medium that can be used to carry or store desired program code in the form of instructions or data structures and that can be accessed by a processor of a general purpose computer.

In various aspects, the systems and methods described in the present disclosure can be addressed in terms of modules. The term “module” as used herein refers to a real-world device, component, or arrangement of components implemented using hardware, such as by an application specific integrated circuit (ASIC) or field-programmable gate array (FPGA), for example, or as a combination of hardware and software, such as by a microprocessor system and a set of instructions to implement the module's functionality, which (while being executed) transform the microprocessor system into a special-purpose device. A module, element, or component may also be implemented as a combination of the two, with particular functions facilitated by hardware alone, and other functions facilitated by a combination of hardware and software. In particular implementations, at least a portion, and in some cases, all, of a module, element, or component may be executed on one or more processors of a general purpose computer. Accordingly, each module may be realized in a variety of suitable configurations, and should not be limited to any particular implementation or example herein. An element, or any portion of an element, or any combination of elements may be implemented as a “processing system” that includes one or more processors. When multiple processors are implemented, the multiple processors may perform the functions individually or in combination. One or more processors in a processing system may execute stored instructions, which may be referred to as software, firmware, middleware, microcode, hardware description language, or otherwise, e.g., instructions, instruction sets, code, code segments, program code, programs, subprograms, software components, applications, software applications, software packages, routines, subroutines, objects, executables, threads of execution, procedures, functions, or any combination thereof.

As used herein, the term “patient” refers to an individual, or person, for which an analysis is assessed relating to a potential or actual medical condition or general wellness. A physician refers to a person licensed to practice medicine.

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents. In the interest of clarity, not all of the routine features of the aspects are disclosed herein. It would be appreciated that in the development of any actual implementation of the present disclosure, numerous implementation-specific decisions must be made in order to achieve the developer's specific goals, and these specific goals will vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of the skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. Sets should be interpreted as a set of elements where the elements number one or more. Accordingly, for a set of X, X would include one or more elements. When at least one processor is configured to perform a set of functions, the at least one processor, individually or in any combination, is configured to perform the set of functions. Accordingly, each processor of the at least one processor may be configured to perform a particular subset of the set of functions, where the subset is the full set, a proper subset of the set, or an empty subset of the set. A processor may be referred to as processor circuitry. A memory / memory module may be referred to as memory circuitry.

As used herein, the phrase “based on” shall not be construed as a reference to a closed set of information, one or more conditions, one or more factors, or the like. In other words, the phrase “based on A” (where “A” may be information, a condition, a factor, or the like) shall be construed as “based at least on A” unless specifically recited differently.

The following aspects are illustrative only and may be combined with other aspects or teachings described herein, without limitation.

Aspect 1 is an apparatus for generating an automated medical opinion, comprising: a communication interface to provide a user interface to a user; a data input module configured to collect user medical data via the user interface, wherein the user medical data includes one or more of patient medical history, at least one diagnostic test result, or current symptom information for the user; a pre-processing module configured to structure and anonymize the user medical data for input to a trained model; the trained model that is configured to analyze the user medical data and output a diagnostic assessment including one or more of a medical diagnosis, a recommended medical treatment, or an recommended diagnostic medical test based on the patient medical history, diagnostic tests, and current symptoms received via the user interface; a report generation module configured to compile the diagnostic assessment in a report for display to the user; a feedback loop to receive feedback on the diagnostic assessment from one or more of the user or a medical professional; and a training module that is configured to update the trained model based on one or more of the feedback and new data.

Aspect 2 is a method of reporting an automated medical opinion, including: receiving, via a user interface, one or more of patient medical history, at least one diagnostic test result, or current symptom information for a user; generating, as automated output from a trained model, a diagnostic assessment including one or more of a medical diagnosis, a recommended medical treatment, or an recommended diagnostic medical test based on the patient medical history, diagnostic tests, and current symptoms received via the user interface; sending a report to the user based on the diagnostic assessment; receiving, via a feedback loop, feedback for the diagnostic assessment reported to the user; and updating the trained model based on the feedback.

In aspect 3, the method of aspect 2 further includes that the trained model comprises an artificial intelligence model that is trained for medical diagnostic assessments across multiple medical areas based on a combination of medical cases, medical research, clinical guidelines, and historical patient outcomes.

In aspect 4, the method of aspect 3 further includes receiving, as updated training data becomes available, the updated training data that includes one or more of: additional medical research, updated clinical guidelines, or additional medical cases or patient outcomes; and refining the trained model based on the updated training data.

In aspect 5, the method of aspect 3 or 4 further includes pre-processing patient data that is received via the user interface to standardize and anonymize patient data for input to the artificial intelligence model.

In aspect 6, the method of any of aspects 3-5 further includes that the diagnostic assessment includes one or more inferred medical diagnosis output from the artificial intelligence model.

In aspect 7, the method of any of aspects 3-6 further includes that the diagnostic assessment includes one or more recommended medical treatment inference output from the artificial intelligence model.

In aspect 8, the method of any of aspects 3-7 further includes that the diagnostic assessment includes one or more recommended diagnostic medical test output as an inference from the artificial intelligence model to refine the medical diagnosis or the recommended medical treatment.

In aspect 9, the method of any of aspects 3-8 further includes maintaining a user record with collected information and prior diagnostic assessment; receiving, via the user interface, updated user health information; obtaining an updated diagnostic assessment or wellness recommendation for the user based on the user record and the updated user health information; and sending a notification to the user of the updated diagnostic assessment or the wellness recommendation generated by the trained model.

In aspect 10, the method of any of aspects 3-9 further includes receiving a user subscription to ongoing diagnostic assessments or wellness recommendations.

In aspect 11, the method of any of aspects 3-10 further includes receiving, via the user interface, at least one of the patient medical history or the current symptom information for the user as spoken words via a voice module.

In aspect 12, the method of aspect 11 further includes identifying relevant medical information in the spoken words received via the voice module; and structuring the relevant medical information from the spoken words in a format for entry as input to the trained model to obtain the diagnostic assessment from the trained model.

Aspect 13 is a non-transitory computer-readable medium storing computer executable code for information modeling, the code when executed by processor circuitry causes a medical opinion system to perform the method of any of aspects 2-13.

Aspect 14 is an apparatus for information modeling at a medical opinion system, comprising memory and one or more processors configured to cause the medical opinion system to perform the method of any of aspects 2-13.

Aspect 15 is a processing system for a medical opinion system, comprising memory circuitry and processor circuitry coupled to memory circuitry, and based at least in part on information stored in the memory circuitry, the processor circuitry is configured to cause a medical opinion system to perform the method of any of aspects 2-13.

What is claimed is:

1. An apparatus for generating an automated medical opinion, comprising:
a communication interface to provide a user interface to a user;
a data input module configured to collect user medical data via the user interface, wherein the user medical data includes one or more of patient medical history, at least one diagnostic test result, or current symptom information for the user;
a pre-processing module configured to structure and anonymize the user medical data for input to a trained model;
the trained model that is configured to analyze the user medical data and output a diagnostic assessment including one or more of a medical diagnosis, a recommended medical treatment, or an recommended diagnostic medical test based on the patient medical history, diagnostic tests, and current symptoms received via the user interface;
a report generation module configured to compile the diagnostic assessment in a report for display to the user;
a feedback loop to receive feedback on the diagnostic assessment from one or more of the user or a medical professional; and
a training module that is configured to update the trained model based on one or more of the feedback and new data.

2. A non-transitory computer-readable medium storing computer executable code for reporting an automated medical opinion, the code when executed by processor circuitry causes a medical opinion system to:
receive, via a user interface, one or more of patient medical history, at least one diagnostic test result, or current symptom information for a user;
generate, as automated output from a trained model, a diagnostic assessment including one or more of a medical diagnosis, a recommended medical treatment, or an recommended diagnostic medical test based on the patient medical history, diagnostic tests, and current symptoms received via the user interface;
send a report to the user based on the diagnostic assessment;
receive, via a feedback loop, feedback for the diagnostic assessment reported to the user; and
update the trained model based on the feedback.

3. The non-transitory computer-readable medium of claim 2, wherein the feedback is received from the user.

4. The non-transitory computer-readable medium of claim 2, wherein the code when executed by the processor circuitry further causes the medical opinion system to send the diagnostic assessment to one or more medical professionals, wherein the feedback is received from the one or more medical professionals indicating an adjustment to the diagnostic assessment.

5. The non-transitory computer-readable medium of claim 2, wherein the trained model comprises an artificial intelligence model.

6. The non-transitory computer-readable medium of claim 5, wherein the artificial intelligence model is trained for medical diagnostic assessments across multiple medical areas based on a combination of medical cases, medical research, clinical guidelines, and historical patient outcomes.

7. The non-transitory computer-readable medium of claim 6, wherein the code when executed by the processor circuitry further causes the medical opinion system to:
receive, as updated training data becomes available, the updated training data that includes one or more of:
additional medical research,
updated clinical guidelines, or
additional medical cases or patient outcomes; and
refine the model based on the updated training data.

8. The non-transitory computer-readable medium of claim 5, wherein the code when executed by the processor circuitry further causes the medical opinion system to pre-process patient data that is received via the user interface to standardize and anonymize the patient data for input to the artificial intelligence model.

9. The non-transitory computer-readable medium of claim 5, wherein the diagnostic assessment includes one or more inferred medical diagnosis output from the artificial intelligence model.

10. The non-transitory computer-readable medium of claim 5, wherein the diagnostic assessment includes one or more recommended medical treatment inference output from the artificial intelligence model.

11. The non-transitory computer-readable medium of claim 5, wherein the diagnostic assessment includes one or more recommended diagnostic medical test output as an inference from the artificial intelligence model to refine the medical diagnosis or the recommended medical treatment.

12. The non-transitory computer-readable medium of claim 5, wherein the code when executed by the processor circuitry further causes the medical opinion system to:
maintain a user record with collected information and prior diagnostic assessment;
receive, via the user interface, updated user health information;
generate an updated diagnostic assessment or wellness recommendation for the user from the trained model based on the user record and the updated user health information; and
send a notification to the user of the updated diagnostic assessment or the wellness recommendation generated by the trained model.

13. The non-transitory computer-readable medium of claim 12, wherein the code when executed by the processor circuitry further causes the medical opinion system to:
receive a user subscription to ongoing diagnostic assessments or wellness recommendations.

14. The non-transitory computer-readable medium of claim 2, wherein the user interface is configured to receive at least one of the patient medical history or the current symptom information for the user as spoken words via a voice module.

15. The non-transitory computer-readable medium of claim 14, wherein the code when executed by the processor circuitry causes the medical opinion system to:
identify relevant medical information in the spoken words received via the voice module; and
structure the relevant medical information from the spoken words in a format for entry as input to the trained model to obtain the diagnostic assessment from the trained model.

16. A method of reporting an automated medical opinion, including:
receiving, via a user interface, one or more of patient medical history, at least one diagnostic test result, or current symptom information for a user;
generating, as automated output from a trained model, a diagnostic assessment including one or more of a medical diagnosis, a recommended medical treatment, or an recommended diagnostic medical test based on the patient medical history, diagnostic tests, and current symptoms received via the user interface;
sending a report to the user based on the diagnostic assessment;
receiving, via a feedback loop, feedback for the diagnostic assessment reported to the user; and
updating the trained model based on the feedback.

17. The method of claim 16, wherein the trained model comprises an artificial intelligence model that is trained for medical diagnostic assessments across multiple medical areas based on a combination of medical cases, medical research, clinical guidelines, and historical patient outcomes.

18. The method of claim 17, further comprising:
receiving, as updated training data becomes available, the updated training data that includes one or more of:
additional medical research,
updated clinical guidelines, or
additional medical cases or patient outcomes; and
refining the trained model based on the updated training data.

19. The method of claim 17, further comprising:
pre-processing patient data that is received via the user interface to standardize and anonymize patient data for input to the artificial intelligence model.

20. The method of claim 17, wherein the diagnostic assessment includes one or more inferred medical diagnosis output from the artificial intelligence model.

21. The method of claim 17, wherein the diagnostic assessment includes one or more recommended medical treatment inference output from the artificial intelligence model.

22. The method of claim 17, wherein the diagnostic assessment includes one or more recommended diagnostic medical test output as an inference from the artificial intelligence model to refine the medical diagnosis or the recommended medical treatment.

23. The method of claim 17, further comprising:
maintaining a user record with collected information and prior diagnostic assessment;
receiving, via the user interface, updated user health information;
obtaining an updated diagnostic assessment or wellness recommendation for the user based on the user record and the updated user health information; and
sending a notification to the user of the updated diagnostic assessment or the wellness recommendation generated by the trained model.

24. The method of claim 23, further comprising:
receiving a user subscription to ongoing diagnostic assessments or wellness recommendations.

25. The method of claim 16, further comprising:
receiving, via the user interface, at least one of the patient medical history or the current symptom information for the user as spoken words via a voice module.

26. The method of claim 25, further comprising:
identifying relevant medical information in the spoken words received via the voice module; and
structuring the relevant medical information from the spoken words in a format for entry as input to the trained model to obtain the diagnostic assessment from the trained model.
